(19) United States
(12) Plant Patent Application Publication
(10) Pub. No.: US 2006/0130195 P1
(43) Pub. Date: Jun. 15, 2006

(54) Apple tree rootstock named 'G.202'

(50) MALUS DOMESTICA X ROBUSTA; APPLE ROOTSTOCK

(76) Inventors: James N. Cummins, Geneva, NY (US); Herbert S. Aldwinckle, Geneva, NY (US); Terence L. Robinson, Geneva, NY (US)

Correspondence Address: Michael L. Goldman, Esq.;Nixon Peabody LLP, Clinton Square, P.O. Box 31051, Rochester, NY, 14603, US

(21) Appl. No.: 11/010,982

(22) Filed: Dec. 13, 2004

(51) Int. Cl. A01H 5/00

(52) U.S. Cl. PLT/174

(57) ABSTRACT

The present invention relates to a new and distinct variety of apple tree rootstock named ‘G.202,’ which is a hybrid of a cross between a Malus×domestic cv. Malling 27 apple tree and a Malus×robusta cv. Robusta 5 apple tree. The ‘G.202’ rootstock is a dwarfing rootstock that is resistant to fire blight (caused by Erwinia amylovora) and to crown and root rots (caused by Phytophthora cactorum), and is immune to infestation by the woolly apple aphid (Erisoma lanigerum). The ‘G.202’ apple rootstock is useful in that it can be propagated clonally and used as a rootstock or root system for apple trees, as well as for interstems of apple trees.

### LATIN NAME

Malus×domestica/Malus×robusta hybrid

### VARIETY DENOMINATION

‘G.202’

### BACKGROUND OF THE INVENTION

The present invention relates to a new and distinct variety of apple tree rootstock named ‘G.202.’ ‘G.202’ is botanically classified as a Malus×domestica/Malus×robusta hybrid. ‘G.202’ belongs to the apple tree rootstock market class. Thus, ‘G.202’ is useful in that it can be propagated clonally and used as a disease resistant (i.e., to fire blight, crown rot, and root rot) and insect immune (i.e., to infestation of the woolly apple aphid) rootstock or root system for apple trees, as well as for interstems of apple trees.

### Parental Plants

Malus×domestica cv. Malling 27 (also referred to herein as “Malling 27” or “M.27”) is a super-dwarfing rootstock, i.e., trees grown on this rootstock are 15 to 20 percent the size of a standard self-rooted seedling tree. Malling 27 is known to induce good precocity to the scion (i.e., the ability to induce early reproductive development in the scion), and has high yield efficiency. Malling 27 may induce small fruit size in some scion cultivars. Malling 27 plants produce no spines. In addition, Malling 27 is poorly anchored (i.e., it has shallow roots) and is sensitive to winter cold, drought, and the woolly apple aphid (Erisoma lanigerum).

Malus×robusta cv. Robusta 5 (also referred to herein as “Robusta 5” or “R.5”) is a non-dwarfing rootstock. Robusta 5 does not induce precocity to the scion and is not highly yield efficient. Juvenile plants of Robusta 5 produce many spines. Robusta 5 breaks buds very early in the spring. Robusta 5 is resistant to powdery mildew (caused by Podosphaera leucotricha) and fire blight, and is immune to the woolly apple aphid. Further, Robusta 5 has shown tolerance to the replant disease complex.

### Comparison of the ‘G.202’ Rootstock

The ‘G.202’ apple rootstock of the present invention has a combination of qualities that distinguishes it from its parental plants (i.e., Malling 27 and Robusta 5). For example, although ‘G.202’ has dwarfing properties derived from M.27, unlike its super-dwarfing parent (i.e., M.27), ‘G.202’ produces a tree that is 35 to 40 percent the size of a standard self-rooted seedling tree. Thus, ‘G.202’ belongs to a different dwarfing (vigor) class than its M.27 parent. In particular, ‘G.202’ has dwarfing characteristics that are similar to those of Malus domestica cv. Malling 26 (“M.26”), and thus belongs to the dwarfing class represented by M.26. While ‘G.202’ is like M.27 in that they both induce precocity to the scion and are highly yield efficient, unlike M.27, ‘G.202’ does not induce small fruit size. With regard to its Robusta 5 parent, ‘G.202’ is distinguishable in that it is a dwarfing rootstock and is highly yield efficient, while Robusta 5 is not. However, like R.5, ‘G.202’ is resistant to fire blight and powdery mildew, and is immune to the woolly apple aphid. In addition, ‘G.202’ has been shown to have tolerance to the replant disease complex, a phenomenon resulting in poor growth when apple trees are replanted in soil where apple trees had been growing previously. The cause of replant disease complex between regions and soils is variable, but usually involves fungal pathogens.

As discussed above, ‘G.202’ is most similar to M.26 in terms of dwarfing class. However, when compared to M.26, ‘G.202’ is more yield efficient than M.26. Further, ‘G.202’ is resistant to fire blight, crown rot, and root rot, and immune to the woolly apple aphid, whereas M.26 and other rootstocks in that class are susceptible to these pathogens and insect pest.

### Origin of the ‘G.202’ Rootstock

In the spring of 1975, pollen from a Robusta 5 apple tree was applied to emasculated flowers of a Malling 27 (referred to herein as “Malling 27” and/or “M27”) apple tree at a greenhouse at the New York State Agricultural Experiment Station (“NYSAES”), Cornell University, Geneva, N.Y. In the fall of 1975, approximately 500 seeds were extracted from mature fruit harvested from plants of this cross. In the winter of 1975 to 1976, the seeds were stratified and planted in large flats under conditions effective to germinate seedlings from the seeds. When the germinated seedlings were about 2.5 centimeters (cm) tall, they were inoculated with a mixture of isolates of the fungus Phytophthora cactorum (i.e., the causal agent of crown and root rots of apple). The flats containing the germinated seedlings were flooded to mid-hypocotyl level and kept at 23° C. for one week. The surviving seedlings were then transplanted into individual pots.

In the summer of 1976, the transplanted seedlings were each inoculated with approximately 1×10⁶ colony forming units of the Ea 273 strain of the fire blight bacterium Erwinia amylovora by inserting a 26-gauge hypodermic syringe needle into the shoot tips of the transplanted seedlings. The seedling designated as #202 (from the M.27×R.5 cross made in 1975) was labeled “75M27R5-202” and was one of 45 surviving seedlings of the above-described battery of inoculations from the same cross. In the fall of 1976, the 45 surviving seedlings were transplanted to the Loomis Farm (NYSAES, Cornell University, Geneva, N.Y.) and allowed to grow side shoots for propagation/evaluation. In 1978, 75M27R5-202 (i.e., ‘G.202’) was evaluated for rooting ability in a layering bed (i.e., a stool bed), lack of spine production, and low root brittleness. In 1982, four finished trees with the 75M27R5-202 rootstock were planted in a first test orchard on the Loomis farm (NYSAES, Cornell University, Geneva, N.Y.) with Northern Spy (Malus×domestica) grafted onto this rootstock as the scion cultivar. In 1983, two trees of the ‘G.202’ rootstock were also planted with Golden Delicious (Malus×domestica) as the scion cultivar. This rootstock performed well (i.e., in the top 20 percent of many rootstocks tested) in these first test trials and more material was propagated by stool bed to be entered into new trials at the Loomis farm (NYSAES, Cornell University, Geneva, N.Y.). The ‘G.202’ rootstock performed well with all the scion cultivars that were tested. The excellent field performance in stool beds as well as in the orchard as rootstocks on finished trees warranted the endorsement for commercialization as apple rootstock ‘Geneva 202’ (i.e., ‘G.202’) by the inventors.

### Asexual Reproduction of the ‘G.202’ Apple Rootstock

Asexual reproduction of the ‘G.202’ apple rootstock has been achieved using the traditional method of clonally propagating apple rootstocks. In particular, the original seedling of the ‘G.202’ apple rootstock was planted in the Loomis farm (NYSAES, Cornell University, Geneva, N.Y.) and allowed to develop into a “mother plant.” The ‘G.202’ mother plant was then used to obtain rooted liners using conventional layering procedures. The resulting liners were then planted in a row to generate a layering stool bed (also referred to as the “mother stool bed”). The living tissues (i.e., the leaves, stems, buds, and spines) of the mother stool bed were observed and determined to be identical to secondary and tertiary stool bed plants. In addition to conventional layering, the ‘G.202’ apple rootstock variety has been asexually reproduced by root cuttings and by budding and grafting onto seedling and clonal rootstocks.

The asexual propagules of the ‘G.202’ apple rootstock resulting from the above-described asexual propagation methods have remained true-to-type.

### BRIEF SUMMARY OF THE INVENTION

The present invention relates to a new and distinct variety of apple tree rootstock named ‘G.202.’ The ‘G.202” rootstock is a dwarfing rootstock that is resistant to fire blight (caused by Erwinia amylovora) and crown and root rot (caused by Phytophthora cactorum), and is immune to infestation by the woolly apple aphid (Erisoma lanigerum). The ‘G.202’ apple rootstock is useful in that it can be propagated clonally and used as a rootstock or root system for apple trees, as well as for interstems of apple trees.

While ‘G.202’ rootstock is a hybrid of a cross between Malling 27 and Robusta 5, ‘G.202’ is distinct from its parent cultivars in terms of dwarfing. In particular, ‘G.202’ shows less dwarfing than super-dwarfing parent Malling 27, but more dwarfing that non-dwarfing parent Robusta 5. Further, unlike Malling 27, ‘G.202’ is not poorly anchored and is not susceptible to the woolly apple aphid. Unlike Robusta 5, ‘G.202’ is resistant to root rot and crown rot. Although ‘G.202’ has been shown to belong to the dwarfing class of M.26, ‘G.202’ is also distinguishable from M.26. In particular, unlike ‘G.202,’ M.26 is susceptible to fire blight, crown rot, root rot, and woolly apple aphid.

### BRIEF DESCRIPTION OF THE DRAWINGS

FIG. 1 is a photograph showing a dormant shoot (including buds and bark) of the new variety of apple rootstock named ‘G.202.’

FIG. 2 is a photograph showing the adaxial (upper) lamnia surface of an adult leaf of the new variety of apple rootstock named ‘G.202.’

FIG. 3 is a photograph showing flowers of the new variety of apple rootstock named ‘G.202.’

FIG. 4 is a photograph showing fruits and seeds of the new variety of apple rootstock named ‘G.202.’

### DETAILED BOTANICAL DESCRIPTION

The present invention relates to a new and distinct variety of apple tree rootstock named ‘G.202’ substantially as shown and described herein. The ‘G.202’ apple rootstock exhibits resistance to fire blight (caused by Erwinia amylovora) and to root and crown rots (caused by Phytophthora cactorum), and is immune to infestation by the woolly apple aphid (Erisoma lanigerum). The ‘G.202’ apple rootstock is botanically classified a Malus×domestica/Malus×robusta hybrid. The ‘G.202’ apple rootstock is useful in that it can be propagated clonally and used as a rootstock or root system for apple trees, as well as for interstems of apple trees.

The characteristics of the new and distinct variety of apple rootstock ‘G.202’ are described below. The numerical color specifications used to describe the various characteristics of the ‘G.202’ apple rootstock are those of The Royal Horticultural Society Colour Chart (1976). Botanical descriptions follow the Manual of Cultivated Plants (Bailey, 1949).

### Tree Habit

The unbudded tree of ‘G.202’ is a small shrub, typically standing about 2.0 meters tall by about 2.0 meters wide when 5-years-old. There is no single dominant trunk. Instead, there are from about 3 to 5 shoots arising from the roots. The growth rate of the tree of the ‘G.202’ apple rootstock is moderately slow, with early cessation of shoot growth typically occurring about 1 week earlier than for Malling 26 (i.e., an apple rootstock having similar dwarfing characteristics). Spring budbreak is midseason to moderately late. Very few suckers (i.e., new shoots emerging from below ground) are produced, and, when any suckers are produced, they are not vigorous.

### Productivity

In an intermediate trial performed at the NYSAES (Cornell University, Geneva, N.Y.), the ‘G.202’ rootstock received the cultivar Empire (Malus×domestica) as the scion and was compared to the Malus×domestica check rootstocks M.9 EMLA, M.26, and M.7 with the same scion cultivar. ‘G.202’ was shown to have statistically similar (p=0.05) yield efficiency (kg yield/cm² trunk cross sectional area) to M.9 EMLA.

### Precocity

Scion cultivars budded on ‘G.202’ were shown to exhibit the same precocity as those budded on M.9.

### Fertility (Fecundity)

The ‘G.202’ plant produces flowers and fruits regularly.

### Dormant Shoots (Buds and Bark)

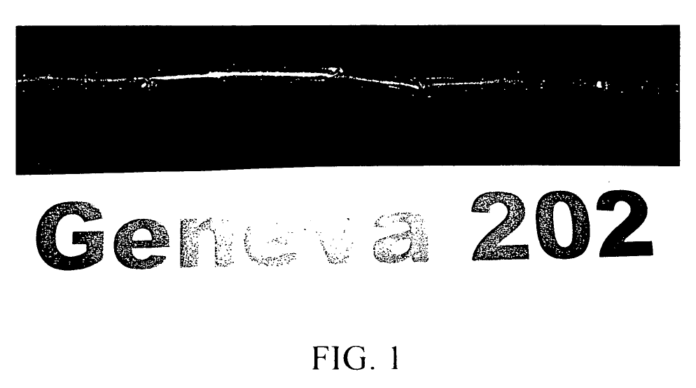

In the stool bed and in the nursery row, dormant mature shoots are Greyed-Red (178B) where exposed to full sunlight (see FIG. 1), grading to Greyed-Orange (171A) with diminished light exposure. Light pubescence is present and gradually disappears in older tissues. The bark on two-year-old shoots is Grey-Brown (199A). Small-sized Greyed-Green (198A) lenticels are present throughout the bark (1-2 lenticels per cm²) and measure on average 0.3-0.4 mm in diameter. In the fall, dormant shoots from the stool bed are 50-80 cm long and may have some spines. Axillary buds are about 4 mm long and 3 mm wide with little pubescence. Axillary buds are obtuse, sessile, and somewhat appressed and flattened.

### Leaves

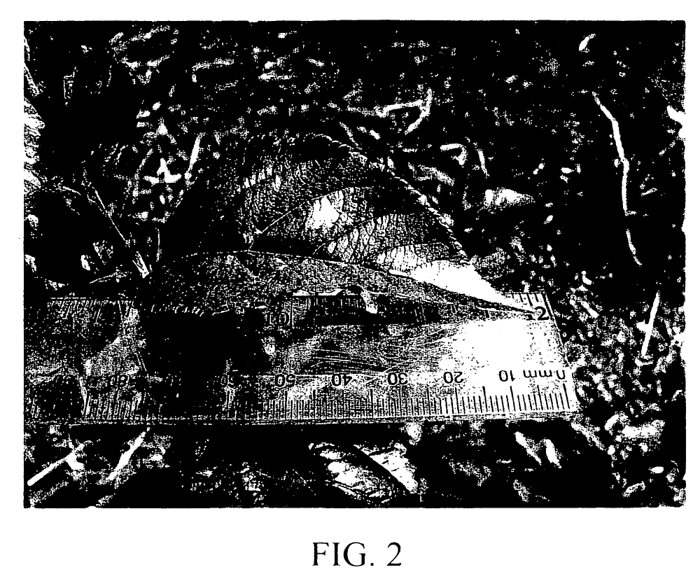

A typical mature leaf of the ‘G.202’ apple rootstock is shown in FIG. 2. The leaves of ‘G.202’ are simple, recurved, and oblong-ovate. The average mature leaf is about 80 mm long and about 50 mm wide (at its widest point). The laminae are somewhat wavy. The leaf tips are acuminate tip. The margins of the leaves are acutely serrated, with about 5 serrations per cm. The leaf has a nearly symmetrical rounded base. Stipules are absent or small (about 1 mm wide and about 7 mm long) and Green (143 B). The leaves have an average petiole length of 22 mm. The petioles are about 2 mm in diameter and Green (141 C) in underbrush. The petioles in young growth exposed to light are colored with a gradation from the base to the lamina—i.e., from Greyed-Red (180A) to Green (141C)—due to production of anthocyanin. The adaxial (upper) lamina surface is Green (137B), translucent, and glabrous. The abaxial (lower) surface is Yellow-Green (146C) and somewhat pubescent. The lamina has a netted venation pattern. Leaf poise is typically 15°-25° from the shoot, depending on shoot orientation. The leaf arrangement is alternate.

### Flowers and Fruit

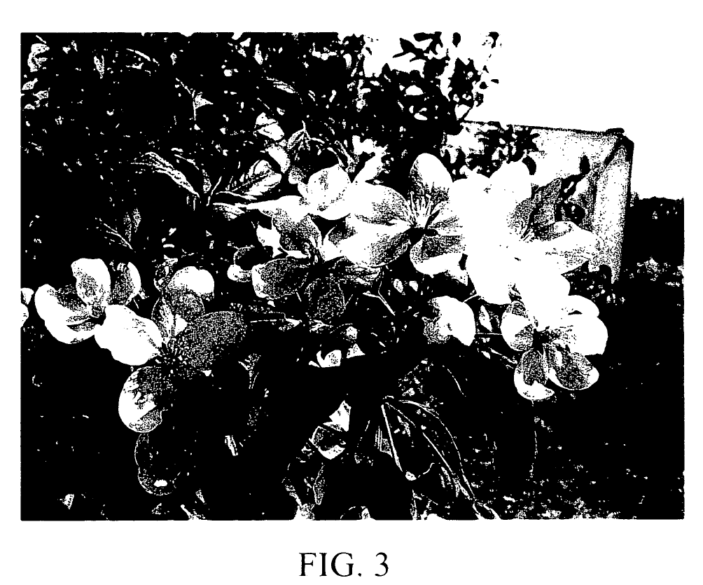

The flowers of the ‘G.202’ apple rootstock variety of the present invention is illustrated in FIG. 3. The flowers of the ‘G.202’ apple rootstock variety are borne on spurs, on shoot terminals, and from lateral buds on growth from the previous season. Buds on spurs and terminals are mixed, typically producing a truss of 5 to 6 flowers and one bourse shoot. The plant flowers regularly every year. Lateral buds are obtuse, sessile, and somewhat appressed, and are about 4 mm long by about 3 mm wide. Buds near the base of the shoot of the previous season usually produce 3 to 5 flowers and a single short shoot; mid-shoot buds may have 2 to 4 flowers; and more distal buds are usually vegetative. Petals are about 25 mm in length, about 18 mm in width, with spoon (spatulate) shape, obtuse (rounded) apex, smooth margin, and smooth texture. The color of the closed petals is Pink (54A) and of the open petals is White (155D) with Pink (54A) striations showing up in some of the petals. The flower diameter is about 40 mm. The flowers have no characteristic fragrance. The pistils are about 6 mm in length and Yellow-White (145B). The stamen is about 4 mm in length and White (155D). Anther color is Yellow-Brown (167D).

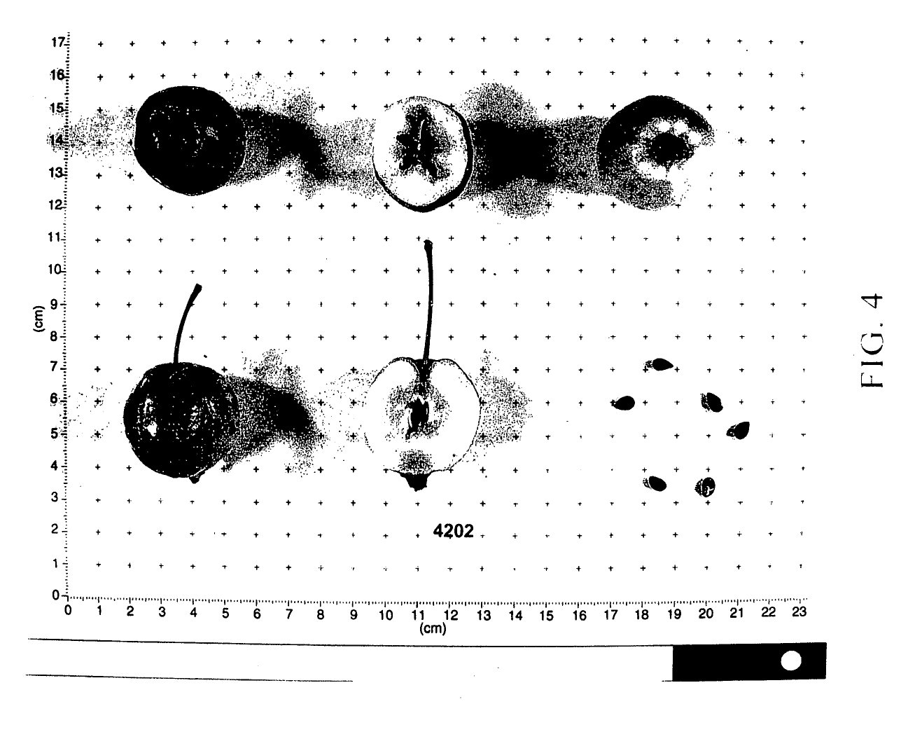

As shown in FIG. 4, the fruit matures into a flat globose shape (obloid) with partial Yellow-Orange (23B) skin and Red (42A) blush overtones. The average size of the fruit is about 3 cm in diameter and about 3 cm in height. The fruit has a protruding persistent calyx. Further, the fruit is very astringent in aroma and taste. The average seed of the present invention is about 0.3 cm in diameter and about 0.5 cm in length. The seeds are shaped as elongated spheres.

### Resistance to Plant Pathogens and Immunity to Insect Infestation

As discussed above, the ‘G.202’ apple tree rootstock of the present invention exhibits resistance to the fire blight, crown rot, and root rot and exhibits immunity to infestation by the woolly apple aphid.

What is claimed:

1. A new and distinct variety of apple tree rootstock named ‘G.202’ substantially as shown and described herein.
